(19) United States
(12) Patent Application Publication
(10) Pub. No.: US 2016/0038326 A1
(43) Pub. Date: Feb. 11, 2016

(54) NOVEL COMPRESSION BAND

(71) Applicant: CIZETA MEDICALI FRANCE, St Armand Montrond (FR)

(72) Inventors: Christian GRADON-MOLLARD, Chamalieres (FR); Jean-Francois Vincent CREBASSA, Baillargues (FR); Pierre-Marie NOEL DE LA PAQUERIE, Beziers (FR)

(73) Assignee: Cizeta Medicali France, Saint Armand-montrond (FR)

(21) Appl. No.: 14/782,560

(22) PCT Filed: Feb. 24, 2014
(86) PCT No.: PCT/FR2014/050375

§ 371 Date: Oct. 5, 2015

(30) Foreign Application Priority Data
Apr. 5, 2013 (FR) 1353058

(51) Int. Cl. A61F 5/01; A61F 13/00; A61F 13/08

(52) U.S. Cl. CPC A61F 5/0104; A61F 5/0109; A61F 13/085; A61F 13/00038

(57) ABSTRACT

The present invention relates to a compression band intended for being wound around a lower or upper limb, Specifically, the band includes at least one first (1) and one second (2) area having first and second longitudinal elasticities, respectively, the first and second areas being juxtaposed longitudinally, and a retaining means (3, 4) being provided in order to place the ends of said band vertically on lop of one another in an adjustable manner, such as to adjust the compressive stress applied to said member.

### TECHNICAL FIELD OF THE INVENTION

The invention relates to the field of compression means that can be applied to limbs of a person, in order in particular to hold and/or compress all or part of the limb. More particularly the present invention relates to elastic venous compression orthoses, which are in particular recommended in various clinical manifestations of venous insufficiency of the lower limbs.

### PRIOR ART

The known orthoses are intended to produce a therapeutic effect by compression of the limb to which they are applied, over a greater or lesser extent and with pressures differentiated according to the areas concerned. These orthoses are textile elements, well known by the term “compression stocking” or “compression tights”. These are in fact, most of the time, tubular textile elements that surround and compress a leg. There exist several types of compression stocking, classified according to the pressure that they exert on the limb concerned. Thus, in France, class 1 concerns stockings exerting a pressure of between 10 and 15 mmHg on the ankle; this class is recommended in the case of heavy legs, evening oedema or reticular varices. Class 2 relates to stockings or tights exerting a pressure of between 15 and 20 mmHg on the limb; this class is recommended for larger varices than those in class 1, that is to say greater than 3 mm. Class 3 relates to textiles exerting a pressure of between 20 and 36 mmHg, that is to say for the treatment of permanent oedemata, trophic disorders or healed ulcers.

The known textile elements cited above make it possible to treat, by compression in particular, venous problems, or more precisely pathological manifestations of the venous system, from simple functional disorders to trophic disorders. Elastic stockings are often considered to be a decisive element in therapy against chronic venous insufficiently. By thus compressing the external part of the leg, the veins regain a diameter that allows correct functioning for them. The compression is the effect produced by an elastic orthosis on a limb at rest or at work. The return forces of the elastic fibres of the compression means act almost constantly on the limb concerned.

In other cases, when it is a question of exerting a stronger pressure different from the one mentioned above, holding is spoken of and the means used act differently; it is a case essentially of holding part of the body in a given posture. The pressures involve are around 70 to 80 mmHg and the means used act in particular directly on the sclerotic veins that are compressed in order to reduce pain. At rest the holding means exerts a low or even zero pressure on the limb in question; during muscular contraction the holding means opposes the increase in the volume of muscle, which thus comes to abut on the holding means, which increases the pressure force. The holding is therefore effective and active at work and almost inactive at rest.

It will be understood that, in the case of holding, the means and techniques used go beyond simple stockings or tights.

Thus, through the document FR 2 961 389, a compression/holding orthosis is known, comprising a compressive part in the form of a sock or stocking, associated with the holding part in the form of a non-elastic tubular band placed in the region of the calf, where it is superimposed on the compressive sock. The dimensions of the holding band are adjusted to the dimensions of the calf so as to come into non-holding contact with it in the absence of contraction of the calf muscle. Moreover, the compressive stocking is designed so that it exerts a lower level of pressure in the region of the holding band. This document therefore discloses an orthosis exerting different pressures according to the areas concerned.

A very similar solution is disclosed in the document FR 2 967 051, according to which a sock has elasticated knitting zones that are different from the foot as far as the bottom of the knee. More precisely, this orthosis/sock comprises an elastic compressive distal part and an adjacent holding proximal part, enclosing the region of the calf. The holding part is tubular, essentially deformable, non-elastic and knitted so as to be continuous with the compressive part.

The document WO 2004/000183 is also known, which describes a tubular sleeve having an anterior part made from a non-extensible material, coming into abutment against the tibial crest, and an extensible posterior part enclosing the region to be compressed. These two parts are connected along two connecting generatrices. Small inflatable balloons are moreover interposed between the non-extensible material and the tibial crest; the small balloons can be inflated in a differentiated manner in order to in the end exert a controlled pressure on the region concerned. This solution is relatively sophisticated since a microprocessor is associated with a tubular sleeve, in particular for purposes of individual control of the pressure of the small balloons.

A compressive tubular orthosis is also known from the document WO 02/019955, wherein the mesh or fabric is chosen according to the perimeter dimensions of the lower limb, so as to apply thereto a substantially constant holding pressure over the entire height of the leg. More specifically, the tubular part extends from the foot as far as the bottom of the thigh and comprises an extensible internal region with high elasticity, connected along two generatrices to an external region that is only slightly extensible. Moreover, a sleeve open along a generatrix is provided for surrounding and reinforcing the pressure at the thigh; it is provided with adjustable closure means.

An orthosis of the knitted sock type is also known, comprising a plurality of zones exerting different pressures on various regions of the limb concerned. The document WO 2006/1174459 describes an example of this type of solution.

The known solutions disclosed above are specifically suited to one morphology or they make it possible to produce a given therapeutic effect suited to the case envisaged. A universal solution adaptable to several morphologies or to various pathologies is not known.

Moreover, the known means may be tricky or take a long time to place on a limb, in particular by patients who are clumsy, little trained, aged or afflicted by osteoarthritic or other phenomena. Since the known solutions are in the majority of cases implemented using a knitted mesh, they are sized and produced so as to deliver a given pressure, maximum at the ankle. They therefore by nature have an adjusted shape and an appropriate elasticity which partly explain a difficulty in fitting by the user.

The patent U.S. Pat. No. 5,472,413 is also known, which discloses a splint for a joint, such as for the knee and elbow. This splint comprises a base element and an element in a spider shape. As can be seen in the figures, the spider-shaped element is placed on top of the base element. Consequently said elements are not longitudinally juxtaposed. Furthermore, the base and the spider-shaped element are each formed from a unitary flat sheet made from elastomer material. As a result the elasticity in these sheets is constant over the length of the splint. In addition, this prior invention seeks to cover not a limb but the joint of a limb, which is different from the present invention. Moreover, in this document, it is not proposed to juxtapose two areas with different elasticities.

The prior art moreover comprises the patent application U.S. 2003/0204156, which discloses a bandage consisting of an elastic sleeve made from an elastic material; this document further describes bands superimposed on the bandage. This document does not propose juxtaposing two areas with different elasticities, unlike the subject matter of the present invention. Furthermore, the objective is different.

The patent application U.S. 2012/0179084 is also known, which relates to a compression garment able to be cut and sewn to measure according to the morphology of the patient. Measurement indications are provided and the garment is then cut and attached. Thus this document discloses a compression garment in several textile parts, the body of which is either in a single piece with constant elasticity or in several pieces with a separate connection element. In addition, the garment is subsequently necessarily cut out, unlike the band according to the invention. The objective and the problem stated in this document are remote from those of the present invention.

The prior art moreover comprises the patent U.S. Pat. No. 5,415,624, which describes a bandage provided with pockets able to receive elements containing a liquid.

These elements are placed on top of the bandage rather than juxtaposed therewith. In addition, the liquid is intended to be heated or cooled, which constitutes an objective different from that of the invention. Furthermore, the bandage is itself a body made from a typical elastic material.

### DISCLOSURE OF THE INVENTION

The invention aims to remedy the drawbacks of the prior art and in particular to produce a simple and effective solution to the stated problem. Furthermore, the invention relates to a solution that is technologically easy to implement.

To do this, a compression band intended to be wound around a lower or upper limb is proposed according to a first aspect of the invention; the band comprises at least first and second areas having respectively a first and second longitudinal elasticity, the first and second areas being longitudinally juxtaposed, and a retaining means being provided in order to place ends of said band on top of each other in an adjustable manner, so as to adjust the compression tension applied to said limb. Advantageously, the band is made of a textile material.

Such a band can easily be fitted by the patient himself, without any external assistance. In addition, the patient can easily himself adjust the pressure exerted, by modifying for example the overlap of the ends of the band. The patient can place each specific area of the band precisely on the surface of the limb to be treated. The actions for making this placement are very simple.

According to one embodiment of the invention, the retaining means comprises a fastening surface of the Velcro type at a first end, which cooperates with a conjugate fastening surface disposed at a second end of said band.

Advantageously, say retaining means comprises at least one longitudinal extension of said band. Such an extension is attached to a side forming the width of the band.

According to a particular embodiment of the invention, the band comprises two extensions juxtaposed across the width of the band and attached to a first end of said band, said extensions facilitating gripping of said band in particular for opening thereof. Such an arrangement therefore allows a double function for the extension or extensions: the retaining of the band on itself and the gripping of one end of the band for purposes of manipulation.

In accordance with a particular embodiment of the invention, the band comprises a first area juxtaposed with a second area.

According to another embodiment, the band comprises a first area longitudinally surrounded by two second areas, the first area having an elasticity less than each second area.

Advantageously, the band may further comprise at least one pocket formed at the first area. A rigid element can be provided in order to be inserted in said pouch. Such an arrangement makes it possible to increase the pressure locally; according to the form and dimension of the rigid element, an adjustment and/or precision on this location can be achieved. In addition the rigidity of the rigid element can be chosen according to the case in question.

Moreover, the band according to the invention may comprise at least one stiffening rod oriented substantially across the width of the band and preferentially disposed at at least a longitudinal end of said band. This rod or these rods are preferentially inserted in a slot formed for example by a double seam on the side concerned. They may be put in place, or not, by the user himself.

### BRIEF DESCRIPTION OF THE FIGURES

Other features, details and advantages of the invention will emerge from a reading of the following description with reference to the accompanying figures, which illustrate:

FIG. 1, a front view of the band according to a first embodiment of the invention; and

FIG. 2 a front view of the band according to a second embodiment of the invention.

For more clarity, identical or similar elements are marked by identical reference signs in all the figures.

### DETAILED DESCRIPTION OF AN EMBODIMENT

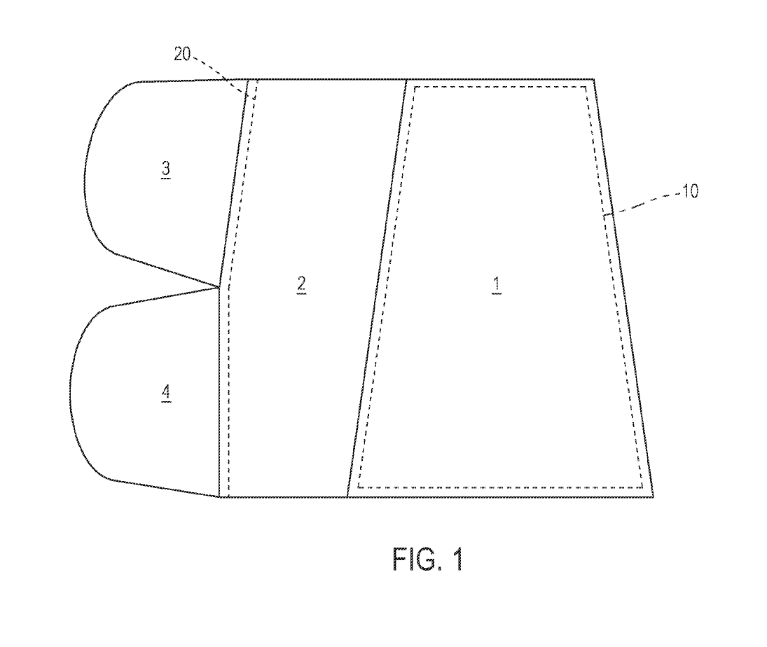

FIG. 1 illustrates schematically a compression band according to a first embodiment of the invention, produced from a textile material. The band comprises a first area longitudinally juxtaposed with a second area 2. The first area 1 has a first elasticity different from that of the second area 2. The first area 1 may consist of an attached textile, for example stitched, and partially overlapping a textile piece, the non-covered surface of which corresponds to the second area 2, which has an elasticity different from that of the first area. The dotted lines 10 correspond to the lines where the first area 1 is stitched on the second area 2. Naturally this embodiment is illustrative and in no way limitative.

The first area 1, the more rigid, can be produced from a knitted textile mesh with a very tight texture. According a preferred embodiment of the invention, the first area is rigid, that is to say non-elastic; the second area 2 has a certain elasticity: for example with a test piece 50 mm wide and 150 mm long, an extension of 125% is obtained under a traction of 74 N. These tests were carried out in accordance with EN 14707-3:2006 Met A.

In addition, the second area 2 may comprise another stitching line 20 that makes it possible to attach at least one retaining means 3, 4 to one of its sides. Here two retaining means 3, 4 are provided. The retaining means is or are in particular intended to place, in a way that can be set and adjusted, the longitudinal ends of the band one on top of the other. The retaining means 3, 4 constitute a kind of longitudinal extension of the first 1 and second 2 areas, advantageously used for gripping and manipulating the band; the means 3, 4 are further used as a means for fastening the first end of the band on the second end of the band. Thus at least one retaining means 3, 4 can have a face provided with fastening elements able to cooperate with the opposite face of the second longitudinal end of the band. To do this the second end is provided with conjugate fastening elements. For example, faces of the Velcro® type can be provided for effecting this adjustable superimposition. Naturally a series of hooks cooperating with rings can be chosen without departing from the scope of the invention. Any technical means can be used.

If two extensions 3, 4 as depicted in FIG. 1 are chosen, disposed on either side of the main longitudinal axis of the band, a certain equilibrium is achieved. The two extensions are juxtaposed according across the width of the band and attached to a first end of the band, for example by the stitching lines 20.

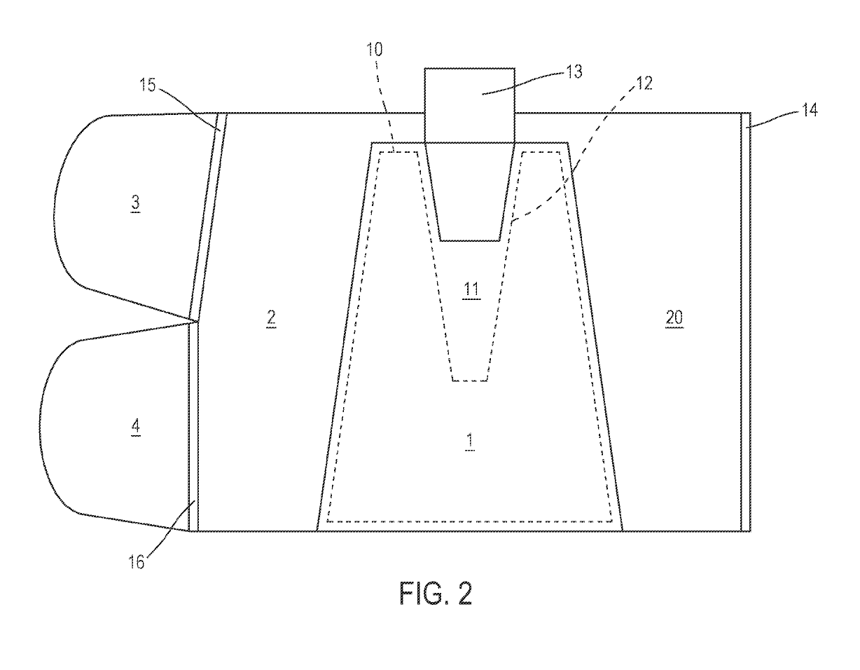

Another embodiment of the invention is shown in FIG. 2. According to this alternative, the band comprises a first area 1 longitudinally surrounded by two second areas 2, 20. The first area 1 is has a rigidity greater than those of the second areas 2, 20, which themselves may have a rigidity that is equal or not.

A pocket 11 formed by means of a specific stitching line 12 between the first area 1 and the second area 2 is also shown schematically in FIG. 2. At least one rigid element 13, for example made from foam, intended to increase the pressure in a specific, controlled and localised manner, can be inserted in this pocket 11. A person skilled in the art will choose the dimensions, forms and rigidities of the rigid element 13 according to the effects to be produced and the constraints (economic, ergonomic, compliance with standards or other) to be complied with.

Naturally such a pocket may be provided in all embodiments of the invention, in particular the one in FIG. 1.

Furthermore the band according the invention may comprise at least one stiffening rod 14, 15, 16 preferentially oriented substantially across the width of the band and at at least one longitudinal end of the band. The rod or rods 14, 15, 16 may be housed in a double seam formed at these locations and providing an opening at one end.

This arrangement allows modulation in order to adapt the band to various configurations.

The pressures exerted on wearers of the band according to the invention have been recorded. Pressures on the calves of five wearers were tested. The pressure on the calf at rest was first of all recorded on the five wearers, and then the pressure in a working state was recorded. These measurements were moreover taken at the rigid part (the first area) of the band, level with the elastic part (the second area) of the band and level with the tibial crest of the wearer. The results show that, at the rigid area 1, the mean pressure varies between 64 and 83 mmHg between these two states, which represents a high increase in pressure on the wearer at this point. At the elastic area 2, the increase in pressure is almost zero (0.2).

Furthermore the tibial crest must not be subjected to an excessive pressure, and the results showed a maximum pressure of less than 60 mmHg. These results are very interesting since the effect sought, namely a differentiated pressure, according to various areas, is indeed obtained by means of the band according to the invention.

Moreover it was observed that the mean variation in the pressure between rest and work is 18 mmHg on the rigid part 1, with a stretching of the band at 30%, 50% and 100%. A considerable improvement in terms of comfort for the wearer is thus obtained according to invention.

1. A compression band intended to be wound around a lower or upper limb, comprising at least first and second areas, and a retaining means being provided in order to place the ends of said band one on top of the other in an adjustable fashion so as to adjust the compression tension applied to said limb, wherein the band is made of a textile material and said at least one first and second areas have respectively a first and second longitudinal elasticity, the first and second areas being longitudinally juxtaposed.

2. A compression band according to claim 1, wherein said retaining means comprises a fastening surface of the Velcro type at a first end, which cooperates with a conjugate attachment surface disposed at a second end of said band.

3. A compression band according to claim 2, wherein said retaining means comprises at least one longitudinal extension of said band.

4. A compression band according to claim 3, wherein at least one longitudinal extension is attached to a side forming the width of said band.

5. A compression band according to claim 4, wherein the at least one longitudinal extension comprises two extensions juxtaposed in the width of the band and attached to a first end of said band, said extensions facilitating the gripping of said band in particular for its opening thereof.

6. A compression band according to claim 4, comprising a first area juxtaposed with a second area.

7. A compression band according to claim 1, comprising a first area longitudinally surrounded by two second areas, the first area having an elasticity less than each second area.

8. A compression band according to claim 1, further comprising at least one pocket formed at the first area.

9. A compression band according to claim 8, further comprising at least one rigid element inserted in said at least one pocket.

10. A compression band according to claim 1, comprising at least one stiffening rod oriented substantially across the width of the band and preferentially disposed at at least one longitudinal end of said band.
